(19) United States
(12) Patent Application Publication
(10) Pub. No.: US 2025/0100875 A1
(43) Pub. Date: Mar. 27, 2025

(54) A METHOD OF FORMING A SYNGAS FOR PRODUCING LIQUID HYDROCARBONS

(71) Applicant: Johnson Matthey Davy Technologies Limited, London (GB)

(72) Inventors: Stuart William ALLAN, Stockton-on-Tees (GB); Andrew James COE, London (GB); Amelia Lorna Solveig COOK, London (GB); Cuijie JIANG, Billingham Cleveland (GB); Raul LLORENS NAVARRO, Billingham Cleveland (GB); Michiel NIJEMEISLAND, Billingham Cleveland (GB); Jon-Pierre SUNDERLAND, Stockton-on-Tees (GB)

(21) Appl. No.: 18/832,719

(22) PCT Filed: Mar. 16, 2023
(86) PCT No.: PCT/GB2023/050627

§ 371 Date: Jul. 24, 2024

(30) Foreign Application Priority Data
Apr. 1, 2022 (GB) 2204767.4

(51) Int. Cl. C01B 3/12; B01D 53/04; B01D 53/14; C01B 32/40; C07C 1/04

(52) U.S. Cl. CPC C01B 3/12; B01D 53/04; B01D 53/1462; C01B 32/40; C07C 1/044; B01D 2252/204; B01D 2253/112; B01D 2257/304; B01D 2257/504; C01B 2203/0415; C01B 2203/0475; C01B 2203/0485; C01B 2203/062; C07C 2523/46; C07C 2523/745; C07C 2523/75

(57) ABSTRACT

A method of forming a syngas for producing liquid hydrocarbons, the method comprising: providing a feed gas comprising carbon dioxide, hydrogen and compounds of sulfur; providing a carbon-monoxide-enriched feed gas by passing the feed gas to a reverse-water-gas-shift reaction chamber to convert a portion of the carbon dioxide and a portion of the hydrogen to carbon monoxide and water, and to convert at least a portion of the compounds of sulfur to hydrogen sulfide; passing the carbon-monoxide-enriched feed gas to a carbon-dioxide-removal unit to provide the syngas and a carbon-dioxide-enriched stream, the carbon-dioxide-enriched stream comprising carbon dioxide and hydrogen sulfide; providing a purified carbon-dioxide stream by passing the carbon-dioxide-enriched stream to a hydrogen-sulfide-removal unit to remove hydrogen sulfide from the carbon-dioxide-enriched stream; and recycling the purified carbon-dioxide stream into the feed gas.

### FIELD OF THE INVENTION

The invention relates to a method of forming a syngas for producing liquid hydrocarbons, and method of producing liquid hydrocarbons from the syngas.

### BACKGROUND OF THE INVENTION

The Fischer-Tropsch process is a collection of chemical reactions that converts a mixture of carbon monoxide and hydrogen into liquid hydrocarbons. These reactions occur in the presence of metal catalysts, typically at temperatures of 150-300° C. and pressures of one to several tens of atmospheres. The Fischer-Tropsch process involves a series of chemical reactions that produce a variety of hydrocarbons, ideally having the formula (CₙH₂ₙ₊₂). The more useful reactions produce alkanes as follows:

(2n+1)H₂+nCO→CₙH₂ₙ₊₂+nH₂O

where n is typically 1-100, or higher. The formation of methane (n=1) is unwanted. Most of the alkanes produced tend to be straight-chain, suitable to be upgraded to produce middle-distillate fuels such as diesel and jet fuel. In addition to alkane formation, competing reactions give small amounts of alkenes, as well as alcohols and other oxygenated hydrocarbons. Co-produced water is a by-product, which is separated from the products of the Fischer-Tropsch reaction. The Fischer-Tropsch reaction is a highly exothermic reaction due to a standard reaction enthalpy (ΔH) of −165 kJ/mol CO combined.

Synthesis gas (syngas) feed to a Fischer-Tropsch unit can be derived from a number of feedstocks; for example, natural gas via steam reforming and/or auto-thermal reforming, municipal solid waste and biomass via high-temperature gasification or carbon dioxide and hydrogen via a reverse-water-gas shift. The latter source is beneficial since it makes use of carbon dioxide that may have been destined to be released to the atmosphere. Carbon dioxide is typically the product of combustion, and as a result, carbon dioxide sources tend to have high levels of contaminants, such as carbonyl sulfide, hydrogen cyanide, ammonia, hydrogen sulfide and carbonyls, which deactivate Fischer-Tropsch catalysts and need to be removed down to single-digit ppb levels. To maximise the carbon dioxide efficiency of such processes, carbon dioxide that is not converted in the reverse-water-gas shift reactor is recycled. However, such recycling can cause contaminants such as hydrogen sulfide to build up in the recycle loop.

U.S. Pat. No. 6,107,353 describes a method of removing hydrogen cyanide and ammonia from a syngas prior to a hydrocarbon synthesis reaction. The method involves passing the syngas to a catalyst to hydrolyse hydrogen cyanide to ammonia. The hydrolysed gas removed from the hydrolysis zone is then contacted with water in a scrubbing zone to dissolve ammonia.

However, in order to achieve ppb levels of hydrogen cyanide and ammonia, the method requires the use of hydrogen-cyanide-absorption zones prior to entry of the syngas into the Fischer-Tropsch reactor. The use of such hydrogen-cyanide-absorption zones increases the complexity of the method.

U.S. Pat. No. 8,551,218 describes a process for the abatement of carbonyl sulfide, carbon disulfide, metal carbonyl compounds, hydrogen sulfide and hydrogen cyanide, ammonia and arsenic and chlorine compounds from a feed gas comprising the steps of contacting the gas in succession with a first purification agent comprising activated carbon, with a second purification agent comprising alumina, with a third purification agent comprising zinc oxide, with a fourth purification agent comprising a zeolitic material and a fifth purification agent comprising zinc oxide and copper oxide.

The present invention seeks to tackle at least some of the problems associated with the prior art or at least to provide a commercially acceptable alternative solution thereto.

### SUMMARY OF THE INVENTION

One aspect of the present disclosure is directed to a method of forming a syngas for producing liquid hydrocarbons, the method comprising:
providing a feed gas comprising carbon dioxide, hydrogen and compounds of sulfur;
providing a carbon-monoxide-enriched feed gas by passing the feed gas to a reverse-water-gas-shift reaction chamber to convert a portion of the carbon dioxide and a portion of the hydrogen to carbon monoxide and water, and to convert at least a portion of the compounds of sulfur to hydrogen sulfide;
passing the carbon-monoxide-enriched feed gas to a carbon-dioxide-removal unit to provide the syngas and a carbon-dioxide-enriched stream, the carbon-dioxide-enriched stream comprising carbon dioxide and hydrogen sulfide;
providing a purified carbon-dioxide stream by passing the carbon-dioxide-enriched stream to a hydrogen-sulfide-removal unit to remove hydrogen sulfide from the carbon-dioxide-enriched stream; and
recycling the purified carbon-dioxide stream into the feed gas.

Another aspect of the present disclosure is directed to a method of producing liquid hydrocarbons from a syngas, the method comprising:
forming a syngas according to the method described herein; and
passing the syngas through a Fischer-Tropsch reaction chamber to produce a liquid hydrocarbon product.

### BRIEF DESCRIPTION OF THE DRAWINGS

FIG. 1 shows a flow diagram of an example of a method according to the present invention.

### DETAILED DESCRIPTION OF THE INVENTION

In a first aspect, the present disclosure is directed to a method of forming a syngas for producing liquid hydrocarbons, the method comprising:
providing a feed gas comprising carbon dioxide, hydrogen and compounds of sulfur; providing a carbon-monoxide-enriched feed gas by passing the feed gas to a reverse-water-gas-shift reaction chamber to convert a portion of the carbon dioxide and a portion of the hydrogen to carbon monoxide and water, and to convert at least a portion of the compounds of sulfur to hydrogen sulfide;
passing the carbon-monoxide-enriched feed gas to a carbon-dioxide-removal unit to provide the syngas and a carbon-dioxide-enriched stream, the carbon-dioxide-enriched stream comprising carbon dioxide and hydrogen sulfide;
providing a purified carbon-dioxide stream by passing the carbon-dioxide-enriched stream to a hydrogen-sulfide-removal unit to remove hydrogen sulfide from the carbon-dioxide-enriched stream; and
recycling the purified carbon-dioxide stream into the feed gas.

Each aspect or embodiment as defined herein may be combined with any other aspect(s) or embodiment(s) unless clearly indicated to the contrary. In particular, any features indicated as being preferred or advantageous may be combined with any other feature indicated as being preferred or advantageous.

Advantageously, the resulting syngas contains particularly low levels of sulfur compounds, meaning that when used in a Fischer-Tropsch process, poisoning of the Fischer-Tropsch catalyst may be reduced. As a result, the catalyst may need to be regenerated or replaced less frequently, leading to a more efficient process.

The method involves recycling carbon dioxide into the feed gas, thereby increasing the carbon efficiency of the method. The inventors have surprisingly found that such recycling can be carried out without a build-up of hydrogen sulfide in the recycle loop. In conventional methods in which the sulfur removal step is carried out prior to the reverse-water-gas shift reaction chamber, it is common that not all of the sulfur species are removed from the feed gas. For example, certain compounds of sulfur, such as carbonyl sulfide and sulfur dioxide, are not readily adsorbed using conventional sulfur-removal units. Such species will be converted to hydrogen sulfide in the reverse-water-gas-shift reaction chamber, the majority of which will then be removed in the carbon-dioxide-removal/separation unit along with carbon dioxide. Accordingly, the hydrogen sulfide will build up over time in the carbon-dioxide recycle loop. As a result, over time the amount of hydrogen sulfide may build up to levels at which it is not possible for the carbon-dioxide-removal unit to remove all the hydrogen sulfide, leading to increased levels of hydrogen sulfide spilling over into the syngas. In the present invention, since the sulfur removal is carried out in the carbon-dioxide recycle loop rather than prior to the reverse-water-gas-shift reaction chamber, hydrogen sulfide does not build up in the recycle loop. As a result, any spill over of hydrogen sulfide, if any, into the syngas is kept to a minimum.

The term “syngas” or “synthesis gas” as used herein may encompass a gas mixture containing hydrogen and one or both of carbon dioxide and carbon monoxide. In the method of the present invention, the first syngas comprises carbon monoxide (i.e. CO) and hydrogen (i.e. molecular hydrogen H₂).

The term “liquid hydrocarbons” as used herein may encompass species formed of carbon and hydrogen that are liquid at room temperature and pressure. The hydrocarbons typically comprise alkanes, and typically comprise from 5 to 100, or higher, carbon atoms per molecule.

The feed gas comprises carbon dioxide (i.e. CO₂), hydrogen and compounds of sulfur. The syngas may comprise other gases such as, for example, carbon monoxide, water, methane and ammonia, as well as solid species such as, for example, dust and coke. The components of the feed gas may vary depending on its method of manufacture and the starting materials used.

The carbon dioxide may be a by-product of combustion. The carbon dioxide may be derived from flue gas. The carbon dioxide may derive from partial oxidation and/or from a gasifier.

The feed gas is passed to a reverse-water-gas-shift reactor. Reverse-water-gas-shift reactors are known in the art. The reverse-water-gas-shift reactor is preferably an autothermal shift vessel, preferably containing a reverse water-gas shift catalyst. Under the reverse-water-gas-shift reaction, carbon dioxide and hydrogen are converted as follows:

CO₂+H₂⇄CO+H₂O

In the method, a portion of the carbon dioxide and a portion of the hydrogen are converted to carbon monoxide and water. As will be appreciated, not all of the hydrogen is converted. This is because of the need for the syngas to contain hydrogen so as to undergo, for example, a subsequent Fischer-Tropsch reaction.

In the reverse-water-gas-shift reactor, compounds of sulfur are converted to hydrogen sulfide. For examples, sulfur dioxide and carbonyl sulfide may be converted as follows:

SO₂+3H₂→H₂S+2H₂O

COS+H₂O H₂S+CO₂

Preferably, the majority of the compounds of sulfur are converted to hydrogen sulfide, more preferably substantially all of the compounds of sulfur are converted to hydrogen sulfide.

The carbon-monoxide-enriched feed gas is passed to a carbon-dioxide-removal unit. Carbon-dioxide-removal units are known in the art. As discussed above, hydrogen sulfide is absorbed in preference to carbon dioxide in a carbon-dioxide-removal unit.

The carbon-dioxide-enriched stream is passed to a hydrogen-sulfide-removal unit. Hydrogen-sulfide-removal units are known in the art.

The method is typically carried out at elevated pressure, preferably at an absolute pressure of from 1 to 5 IPa.

The hydrogen is preferably generated by the electrolysis of water. Such hydrolysis is preferably carried out using renewable energy. The use of such hydrogen, known as “green hydrogen”, may render the method more environmentally friendly.

The compounds of sulfur preferably comprise one or more of sulfur dioxide, mercaptans, disulfides, thiophenes and carbonyl sulfide. Such species may be particularly poisonous to Fischer-Tropsch catalysts. In addition, such species are difficult to remove using conventional sulfur removal units but are easily converted to hydrogen sulfide in a reverse-water-gas-shift reaction chamber.

The feed gas preferably further comprises compounds of nitrogen, more preferably selected from one or more of amine, hydrogen cyanide, nitric oxide and nitrogen dioxide, and wherein the passing the feed gas to the reverse-water-gas-shift reaction chamber converts a portion of the compounds of nitrogen to hydrogen cyanide and/or ammonia. Such species may be particularly poisonous to Fischer-Tropsch catalysts, particularly cobalt catalysts. In addition, hydrogen cyanide and ammonia are easily removed from the syngas, for example by hydrolysing hydrogen cyanide to ammonia and by removing ammonia using a scrubber.

The reverse-water-gas-shift reaction chamber preferably comprises a catalyst comprising nickel. Such a catalyst is particularly suitable for carrying out a reverse-water-gas-shift reaction at favourable temperatures and pressures and at high conversion rates.

The reverse-water-gas-shift reaction chamber is preferably at a temperature of at least 700° C. Such a temperature may result in a particularly high conversion rate of carbon dioxide and hydrogen to carbon monoxide and water. In addition, such a temperature may be particularly suitable for converting compounds of sulfur to hydrogen sulfide and at a high conversion rate.

The carbon-dioxide-removal unit preferably uses: a liquid chemical absorbent, more preferably selected from one or more of an amine (e.g. an alkyl amine selected from MEA, DEA and MDEA) or an alkali-metal carbonate; and/or a liquid physical absorbent, more preferably selected from one or more of methanol, glycols or glycol ethers. Such absorbents may be particularly suitable for removing carbon dioxide. In addition, such absorbents may absorb hydrogen sulfide in preference to carbon dioxide, meaning that the amounts of hydrogen sulfide spilling over into the syngas, if any, may be particularly low. The liquid chemical absorbent may be part of a scrubber.

The hydrogen-sulfide-removal unit preferably comprises a particulate bed of copper adsorbent, preferably a particulate bed of a copper-hydroxycarbonate adsorbent. Such adsorbents are particularly suitable for absorbing hydrogen sulfide. As a result, any recycling of hydrogen sulfide into the feed gas is kept to a minimum.

The copper-adsorbent bed is preferably at a temperature of from 0 to 150° C., more preferably from 10 to 100° C. Higher temperatures may result in copper in the adsorbent being reduced to elemental copper due to the presence of reducing species such as hydrogen and carbon monoxide. Lower temperatures may result in “wetting” of the catalyst bed.

In a further aspect, the present disclosure is directed to a method of producing liquid hydrocarbons from a syngas, the method comprising:
forming a syngas according to the method of the first aspect; and
passing the syngas through a Fischer-Tropsch reaction chamber to produce a liquid hydrocarbon product.

The advantages and preferable features of the first aspect apply equally to this aspect.

Fischer-Tropsch reaction chambers are known in the art. In view of the particularly low levels of compounds of sulfur in the syngas, poisoning of the Fischer-Tropsch catalyst is reduced in comparison to conventional methods. As a result, the efficiency of the method is improved since regeneration or replacement of the catalyst is not required to be carried out as frequently.

The temperature of the Fischer-Tropsch reaction chamber is preferably from 150° C. to 300° C. Lower temperatures may result in unfavourably low levels of liquid hydrocarbons being generated. Higher temperatures may increase the energy cost of the method without a significant increase in the levels of liquid hydrocarbons being produced.

Passing the syngas through a Fischer-Tropsch reaction chamber to produce a liquid hydrocarbon product preferably comprises contacting the syngas with a catalyst comprising cobalt, iron and/or ruthenium. Such a catalyst may be particularly effective at catalysing Fischer-Tropsch reactions and/or enable the reaction to proceed at favourably low temperatures and/or with high yield.

The molar ratio of hydrogen to carbon monoxide in the syngas is preferably from 1.8 to 2.2, since this is close to the stoichiometric ratio of the Fischer-Tropsch reaction of about 2.

The hydrocarbon products from the Fischer Tropsch reaction chamber are separated from co-produced water and unreacted gases, and then may be converted, for example by hydrocracking, into liquid hydrocarbon fuels.

The syngas comprises preferably hydrogen cyanide and the method further comprises, prior to passing the syngas through a Fischer-Tropsch reaction chamber:
converting at least a portion of the hydrogen cyanide to ammonia to provide a first syngas enriched in ammonia and depleted in hydrogen cyanide; and
passing the first syngas to a scrubber and contacting the first syngas with a scrubbing liquid, whereby at least a portion of ammonia contained in the first syngas is retained in the scrubbing liquid to form a second syngas depleted in ammonia and hydrogen cyanide, the syngas being passed through the Fischer-Tropsch reaction chamber being the second syngas.

Hydrogen cyanide and ammonia may poison a Fischer-Tropsch catalyst. Scrubbers and scrubbing liquids are known in the art. Suitable scrubbing liquids may consist essentially of water. The removal efficiency of ammonia may be improved by increasing residence time in the scrubber or by the increase of surface area of the scrubbing liquid by the use of, for example, trays, structured packing or random packing.

Converting at least a portion of the hydrogen cyanide to ammonia to provide the first syngas preferably comprises catalytic hydrolysis of the hydrogen cyanide with water or steam. Catalytic hydrolysis is a simple way of converting hydrogen cyanide to ammonia since it may be carried out by the addition of water and at relatively low temperatures. Accordingly, the simplicity and efficiency of the method may be improved. The water for the hydrolysis may be either sprayed into the syngas stream before it is fed to the hydrolysis bed or added to the syngas as steam. The equilibrium reaction for this is:

HCN+H₂O⇄NH₃+CO

Following hydrolysis, the syngas is preferably cooled prior to contacting the scrubber, more preferably to a temperature of 40° C. or less, even more preferably to ambient temperature.

The catalytic hydrolysis is preferably carried out at a temperature of greater than 100° C., preferably from 150° C. to 300° C. Lower temperatures may result in unfavourably low levels of hydrolysis. Higher temperatures may increase the energy cost of the method without a significant improvement in the conversion of hydrogen cyanide.

The hydrolysis is preferably carried out using an alumina catalyst, more preferably an activated alumina catalyst. Such catalysts may result in a particularly high conversion rate and/or enable operation at a favourably low temperature.

The first syngas preferably comprises less than 10 ppbv hydrogen cyanide. Such a low level of hydrogen cyanide may result in a particularly low level of poisoning of the Fischer-Tropsch catalyst.

The second syngas preferably comprises less than 10 ppbv ammonia. Such a low level of hydrogen cyanide may result in a particularly low level of poisoning of the Fischer-Tropsch catalyst.

The scrubbing liquid preferably comprises co-produced water. The co-produced water may be separated from the products recovered from the Fischer-Tropsch reaction chamber using conventional separation equipment. In a preferred embodiment, passing the second syngas through the Fischer-Tropsch reaction chamber produces a liquid hydrocarbon product and a co-produced water; and the aqueous scrubbing liquid comprises a co-produced water recovered from the Fischer-Tropsch reaction chamber. In addition, the co-produced water will contain dissolved carbon dioxide from the syngas and also from carbon dioxide generated as a by-product in the Fischer-Tropsch reaction chamber. The ammonia removal capacity of the scrubbing liquid may be improved by the presence of carbon dioxide.

Accordingly, it is easier to reduce the ammonia and hydrogen cyanide contents of the syngas to single-digit ppb levels. As a result, poisoning of the Fischer-Tropsch catalyst is further reduced, meaning that the method is more efficient due to the need to regenerate or replace the Fischer-Tropsch catalyst less regularly. Furthermore, it may be possible to avoid the use of hydrogen-cyanide- and ammonia-absorption beds just prior to the Fischer-Tropsch reaction chamber, thereby resulting in a more simplified method in comparison to conventional methods. The use of co-produced water, rather than having to use another source of water such as boiler feedwater or demineralised water, simplifies the method and reduces the operating cost. In addition, because the co-produced water is recovered from the Fischer-Tropsch reaction chamber, the water is free of poisons. Therefore, the risk of introducing poisons that might deactivate the catalyst is reduced over comparable processes using other sources of scrubbing liquid. The use of co-produced water recovered from the Fischer-Tropsch reaction chamber in the aqueous scrubbing liquid may ensure that no further Fischer-Tropsch catalyst poisons are introduced to the system that may be present in other water streams such as boiler feedwater which is dosed with chemicals.

The scrubbing liquid preferably comprises at least 0.01 mol/L carbon dioxide, more preferably at least 0.02 mol/L carbon dioxide. Such concentrations of carbon dioxide may result in a particularly high ammonia-removal capacity of the scrubbing liquid.

In a preferred embodiment, the scrubbing liquid is preferably saturated with carbon dioxide under the temperature and pressure conditions of the scrubber.

The first syngas preferably comprises hydrogen sulfide and the method further comprises passing the first syngas to a sulfur guard bed upstream of the scrubber to remove hydrogen sulfide from the first syngas prior to passing the first syngas to the scrubber. The presence of hydrogen sulfide in the syngas may result in poisoning of the Fischer-Tropsch catalyst.

The sulfur guard bed preferably comprises zinc oxide. Zinc oxide is particularly effective at removing hydrogen sulfide, for example via the following reaction:

H₂S+ZnO→ZnS+H₂O

The sulfur guard bed is located downstream of the hydrogen-cyanide hydrolysis so as to reduce the formation of toxic zinc cyanide (Zn(CN)₂). Upon discharge and disposal of the adsorbent, exposure of Zn(CN)₂ to moisture would release toxic HCN, causing a hazard to personnel.

The sulfur guard bed preferably operates at a temperature of from 15 to 230° C. Such a temperature may be particularly effective for removing hydrogen sulfide from the syngas.

The method preferably further comprises adding an oxygen-containing gas to the second syngas and passing the second syngas to a carbonyl guard bed to remove metal carbonyls from the second syngas prior to passing the second syngas through the Fischer-Tropsch reaction chamber. The presence of metal carbonyls in the syngas may result in poisoning of the Fischer-Tropsch catalyst. The removal of sulfur species, hydrogen cyanide and ammonia occurs before the addition the oxygen-containing gas in order to avoid generation of SOx and NOx.

The carbonyl guard bed preferably comprises activated carbon. Activated carbon may be particularly suitable for removing carbonyls, for example via catalytic oxidation on the carbon surface:

2Fe(CO)s+1 ½O₂→Fe₂O₃+10CO

Ni(CO)₄+½O₂→NiO+4CO

The carbonyl guard bed preferably operates at a temperature of from 15 to 250° C., more preferably from 50 to 150° C. Higher temperatures may result in side-reactions of the syngas with the deposited metals such as methanation over nickel. Lower temperatures may result in low levels of oxidation of carbonyls.

The method preferably further comprises, after passing the second syngas to the carbonyl guard bed, passing the second syngas to an oxygen guard bed to remove oxygen from the second syngas prior to passing the second syngas through the Fischer-Tropsch reaction chamber. The presence of oxygen in the syngas may result in poisoning of the Fischer-Tropsch catalyst.

The oxygen guard bed preferably comprises a catalyst comprising platinum and/or palladium supported on alumina. Such a catalyst is particularly effective at removing oxygen from the syngas.

The oxygen guard bed preferably operates at a temperature of from 100 to 250° C. Higher temperatures may result in unfavourable side reactions. Lower temperatures may result in only low levels of oxygen being removed.

The invention will now be described in relation to the following non-limiting examples.

### Example

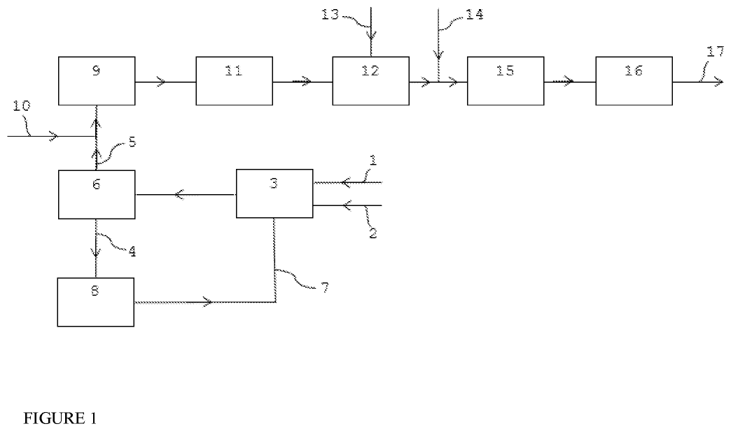

An example of a method according to the present invention is described with reference to FIG. 1. Carbon dioxide 1 and hydrogen 2 feeds react over a nickel catalyst to form carbon monoxide in a Reverse-Water-Gas-Shift (RWGS) Reactor 3 via the following reaction:

CO₂+H₂↔CO+H₂O

Unreacted carbon dioxide 4 is separated from a syngas product stream 5 in a CO₂ Recovery Unit 6 and recycled back via line 7 to the RWGS Reactor feed. H₂S is absorbed with carbon dioxide by the amine in the CO₂ Recovery Unit 6 and the majority will come out in the carbon-dioxide stream 4. If sulfur species such as H₂S and SO₂ are present in the carbon-dioxide feeds, then there is the potential for the build-up of H₂S in the carbon-dioxide recycle loop 7.

SO₂ is converted to H₂S across the RWGS Reactor 3 via the following reaction:

SO₂+3H₂→H₂S+2H₂O

Any COS present in the feed to the RWGS Reactor 3 will hydrolyse to H₂S, which can also then be removed:

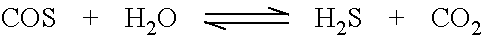

H₂S formed in the carbon dioxide can be readily absorbed, therefore, removal using a bed of copper adsorbent, such as a copper hydroxy-carbonate adsorbent, is located in a CO₂ Sulfur Guard Bed vessel 8 on the carbon-dioxide-recycle loop 7 rather than the fresh carbon-dioxide feed 1.

The carbon dioxide 4 is compressed in a compressor (not shown) and recycled back to the inlet of the RWGS Reactor 3. The CO₂ Sulfur Guard Bed 8 is located after the carbon dioxide stream 4 is compressed as higher pressures reduce the bed size. The gas stream is heated to about 50° C. to prevent the bed being subjected to wetting. This may be achieved by addition of an electric or steam heater or by heat exchange with a hot process fluid. The CO₂ Sulfur Guard Bed vessel 8 captures H₂S and removes it from the circulating carbon dioxide and in this way reduces the amount of H₂S fed to the CO₂ Recovery Unit 6.

The syngas 5 from the CO₂ Recovery Unit 6 is compressed to around an absolute pressure of 5 MPa using a Syngas Compressor (not shown) and undergoes gas clean-up to reduce contaminants to suitable levels to avoid poisoning the downstream Fischer-Tropsch synthesis catalyst. The hot gas leaving the Syngas Compressor is heated to 250° C. via an interchanger, and if desired by using a steam heater or an electric heater. HCN is subsequently hydrolysed to NH₃ over activated high-surface-area alumina in a HCN Hydrolysis Bed 9. A water feed line 10, such as boiler feed water or demineralised water, is provided upstream of HCN Hydrolysis Bed 9 to provide sufficient water for hydrolysis of HCN. The water addition is preferably located upstream of the final heater to generate steam and minimise risk of bed wetting. The HCN Hydrolysis bed 9 is located upstream of a Syngas Sulfur Guard Bed 11 containing a zinc oxide adsorbent to reduce the potential formation of toxic zinc cyanide. Upon discharge and disposal of the zinc oxide adsorbent, exposure of zinc cyanide to moisture could release toxic HCN causing a hazard to personnel.

The hydrolysis reaction is as follows:

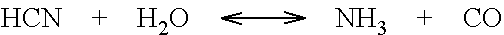

The syngas recovered from the Syngas Sulfur Guard Bed 11 is then cooled in a process interchanger (not shown) before subsequent cooling using cooling water to around 40° C. The water not consumed in the hydrolysis reaction is condensed and is separated in the Syngas Wash Drum 12 and sent to effluent treatment.

Amines and ammonia, either present in the syngas or formed by HCN hydrolysis, are washed from the syngas in the Syngas Wash Drum 12, using FT Co-produced water 13 from the FT Unit (not shown). The Syngas Wash Drum 12 contains trays or packing. The syngas wash water from the Syngas Wash Drum 12 is sent to effluent treatment.

A Carbonyl Guard Bed 15 is provided downstream of the Syngas Wash Drum 12 to remove metal carbonyls from the syngas. An oxygen source 14 is provided upstream of the addition of oxygen 14 to avoid generation of SOx and NOx.

Following the Syngas Wash Drum 12, the syngas stream is heated to 90° C. via a process interchanger (not shown) and then the oxygen 14 is added.

The Carbonyl Guard Bed 15 contains an activated carbon bed. Carbonyls are catalytically oxidized on the carbon surface, for example by reactions as follows:

2Fe(CO)₅+1 ½O₂→Fe₂O₃+10CO

Ni(CO)₄+½O₂→NiO+4CO

The low temperature avoids side reactions of the syngas with the deposited metals.

Surplus oxygen in the syngas recovered from Carbonyl Guard Bed 15 is removed by reaction with components of the syngas in an Oxygen Guard Bed 16 downstream of the Carbonyl Guard Bed 15.

The syngas is heated in a process interchanger (not shown) followed by a steam or electric heater (not shown) to around 150° C. The Oxygen Guard Bed 16 contains platinum or palladium on alumina which catalyses the conversion of oxygen to water in the presence of hydrogen:

O₂+2H₂→2H₂O

A resulting clean syngas stream 17 may then be fed to the FT Unit (not shown).

The foregoing detailed description has been provided by way of explanation and illustration and is not intended to limit the scope of the appended claims. Many variations in the presently preferred embodiments illustrated herein will be apparent to one of ordinary skill in the art and remain within the scope of the appended claims and their equivalents.

1. A method of forming a syngas for producing liquid hydrocarbons, the method comprising:
providing a feed gas comprising carbon dioxide, hydrogen and compounds of sulfur;
providing a carbon-monoxide-enriched feed gas by passing the feed gas to a reverse-water-gas-shift reaction chamber to convert a portion of the carbon dioxide and a portion of the hydrogen to carbon monoxide and water, and to convert at least a portion of the compounds of sulfur to hydrogen sulfide;
passing the carbon-monoxide-enriched feed gas to a carbon-dioxide-removal unit to provide the syngas and a carbon-dioxide-enriched stream, the carbon-dioxide-enriched stream comprising carbon dioxide and hydrogen sulfide;
providing a purified carbon dioxide stream by passing the carbon-dioxide-enriched stream to a hydrogen-sulfide-removal unit to remove hydrogen sulfide from the carbon-dioxide-enriched stream; and
recycling the purified carbon-dioxide stream into the feed gas.

2-7. (canceled)

8. The method of claim 1, wherein the carbon-dioxide-removal unit uses:
a liquid chemical absorbent, preferably selected from one or more of an amine or an alkali-metal carbonate; and/or
a liquid physical absorbent, preferably selected from one or more of methanol, glycols or glycol ethers.

9. The method of claim 1, wherein the hydrogen-sulfide-removal unit comprises a particulate bed of copper adsorbent, preferably a particulate bed comprising a copper-hydroxycarbonate adsorbent.

10. The method of claim 9, wherein the bed is at a temperature of from 0 to 150° C., preferably from 10 to 100° C.

11. A method of producing liquid hydrocarbons from a syngas, the method comprising:
forming a syngas according to the method of claim 1; and
passing the syngas through a Fischer-Tropsch reaction chamber to produce a liquid hydrocarbon product.

12-14. (canceled)

15. The method of claim 11, wherein the syngas comprises hydrogen cyanide and the method further comprises, prior to passing the syngas through a Fischer-Tropsch reaction chamber:
converting at least a portion of the hydrogen cyanide to ammonia to provide a first syngas enriched in ammonia and depleted in hydrogen cyanide; and
passing the first syngas to a scrubber and contacting the first syngas with a scrubbing liquid, whereby at least a portion of ammonia contained in the first syngas is retained in the scrubbing liquid to form a second syngas depleted in ammonia and hydrogen cyanide, the syngas being passed through the Fischer-Tropsch reaction chamber being the second syngas.

16. The method of claim 15, wherein converting at least a portion of the hydrogen cyanide to ammonia to provide the first syngas comprises catalytic hydrolysis of the hydrogen cyanide with water or steam.

17. The method of claim 16, wherein the catalytic hydrolysis is carried out at a temperature of greater than 100° C., preferably from 150° C. to 300° C.

18. The method of claim 16, wherein the hydrolysis is carried out using an alumina catalyst, preferably an activated alumina catalyst.

19-20. (canceled)

21. The method of claim 15, wherein:
passing the syngas through the Fischer-Tropsch reaction chamber produces a liquid hydrocarbon product and co-produced water; and
the scrubbing liquid comprises co-produced water recovered from the Fischer-Tropsch reaction chamber.

22. The method of claim 15, wherein the first syngas comprises hydrogen sulfide and the method further comprises passing the first syngas to a sulfur guard bed upstream of the scrubber to remove hydrogen sulfide from the first syngas prior to passing the first syngas to the scrubber.

23. The method of claim 22, wherein the sulfur guard bed comprises zinc oxide.

24. The method of claim 22, wherein the sulfur guard bed operates at a temperature of from 15 to 230° C.

25. The method of claim 22, wherein the second syngas comprises a metal carbonyl and the method further comprises adding an oxygen-containing gas to the second syngas and passing the second syngas to a carbonyl guard bed to remove the metal carbonyl from the second syngas prior to passing the second syngas through the Fischer-Tropsch reaction chamber.

26. The method of claim 25, wherein the carbonyl guard bed comprises activated carbon.

27. The method of claim 25, wherein the carbonyl guard bed operates at a temperature of from 15 to 250° C., preferably from 50 to 150° C.

28. The method of claim 25, wherein the method further comprises, after passing the second syngas to the carbonyl guard bed, passing the second syngas to an oxygen guard bed to remove oxygen from the second syngas prior to passing the second syngas through the Fischer-Tropsch reaction chamber.

29. The method of claim 28, wherein the oxygen guard bed comprises a catalyst comprising platinum and/or palladium supported on alumina.

30. The method of claim 28, wherein the oxygen guard bed operates at a temperature of from 100 to 250° C.
